(19) United States
(12) Patent Application Publication
(10) Pub. No.: US 2024/0091420 A1
(43) Pub. Date: Mar. 21, 2024

(54) Method and Apparatus for Treating Breast Milk

(71) Applicant: Olle Larsson Holding AG, Zug (CH)

(72) Inventor: Michael Larsson, Zug (CH)

(21) Appl. No.: 18/368,655

(22) Filed: Sep. 15, 2023

(30) Foreign Application Priority Data
Sep. 21, 2022 (DE) 102022124250.1

(51) Int. Cl. A61M 1/06

(52) U.S. Cl. CPC A61M 1/062; A61M 2205/50; A61M 2205/60

(57) ABSTRACT

The present invention relates to a method and an apparatus for treating breast milk, in which a breast pump is applied to the female breast and the breast pump is operated to express and transfer breast milk to a container. The container is sealed, wherein the container is or will be provided with a container identifier that identifies the container. A container identifier identifying the container is generated and assigned to the container. In order to avoid interference with breast milk, it is made available in the container for feeding. The apparatus according to the invention for treating breast milk comprises an accommodation for a container containing breast milk and a closing device for sealing the container, a system for treating the breast milk contained in the container to improve the shelf life of the breast milk, a device for determining the amount of breast milk contained in the container, a reading device for reading out a container identifier provided on the container or attaching device for attaching a container identifier to the container and an interface for transmitting information to a memory characterizing the container identifier and the determined amount of breast milk.

### CROSS-REFERENCE TO RELATED APPLICATION

The present application claims priority to German Application No. 102022124250.1, filed Sep. 21, 2022. The priority application, DE 102022124250.1, is hereby incorporated by reference.

### FIELD OF THE DISCLOSURE

The present invention relates to a method for treating breast milk and an apparatus for treating breast milk.

### BACKGROUND

Premature and very premature infants are usually fed with breast milk. In this context, the breast milk envisaged by the present invention may be a milk as delivered by the woman giving birth to the premature or very premature infant, or by another donor of breast milk.

### DESCRIPTION

The present invention aims to provide a method and an apparatus that enable the hygienically safe and simple donation of breast milk.

In the method according to the invention, breast milk is pumped out with a breast pump which is applied to the female breast and transferred to a container. The container is thereby fluidically connected directly to the breast pump. Thus, the pumped breast milk is transferred directly into the container by the operation of the breast pump. In this way, repeated transfer of breast milk prevents it from being lost or hygienically questionable conditions from arising which could render the breast milk unusable. A container is preferably packed in a sterile or low-germ state, and in this packaging is made available to the donor of the breast milk and in this state is fluidically connected to the breast pump. For this purpose, for example, a flexible bag can be fluidically connected to the breast shield, preferably directly attached to it and coupled to the breast shield. Short conduit paths between the breast and the container are preferable, so that at the end of the breast milk donation, as far as possible the entire delivered volume of breast milk is transferred into the container and only a very small residual volume remains in the conduit path between the female breast and the container. The conduit path into a container formed by the bag within the breast pump can be formed quite predominantly by, for example, grommet-shaped wall segments of the bag itself, which form the entrance into the bag.

The container filled in this way is sealed. Here, too, care must be taken to ensure germ-free or sterile conditions. The closure can be a screw cap, which is handled only on those surfaces that are provided on the outside of the container and do not come into contact with the breast milk. Such a closure is usually supplied to the donor along with the container in the sterile or germ-free package. The container can also be a flexible bag supplied in sterile packaging. The use of a bag as a container has several advantages, which will be discussed further below.

The container can already be provided with a container identifier in the delivered condition, which uniquely identifies the container. Alternatively, the container can also be provided with a corresponding container identifier by the donor or at the location of the donor. The container identifier is saved in an online memory with at least one property of the milk contained in the container. The property of the milk may be, for example, the amount of milk contained in the container. Qualitative properties, such as the time of donation, which indicates the freshness of the breast milk, or even the results of the chemical analysis of the donated breast milk can also be properties of the same. Any medications or nutrients added to the breast milk, especially in the form of fortifiers, can also be saved as a property of the breast milk in the online memory and for the corresponding container.

The same applies to the documentation of a treatment of the breast milk prior to feeding, for example by documenting the treatment step or steps carried out and/or the property or properties of the breast milk modified by the treatment. In particular, the treatment may include filtering, for example to remove toxic ingredients from the breast milk, or concentrating. Concentrating usually involves removing a certain amount of water from the breast milk.

In this context, the container identifier is usually a machine-readable identifier, such as an RFID identifier or a barcode. By reading the container identifier on an input device, for example a PC, the properties saved there for the container can be read out from the online memory. Thus, the properties of the breast milk can be checked, for example, before feeding the breast milk to a premature or very premature infant.

The container identifier is read out at least at a transfer point where the milk contained in the container is stored or consumed. Such a transfer point can be, for example, the goods receipt at a hospital where the premature or very premature infant to be fed is cared for. However, a transfer point can also be, for example, the entrance to a storage facility that is operated centrally or decentrally. Decentralized storage can also take place at the donor's location, for example.

Reading the container identifier and, if applicable, the information saved with it in the online memory about the properties of the breast milk contained in the container allows appropriate storage, timely consumption of the breast milk, and pre-planned feeding of the breast milk to the premature or very premature infant. If the container identifier is read out at a transfer point where the contents of the container are fed, the properties read out with the container identifier can be automatically logged and saved in the form of a nutrition profile for the respective premature or very premature infant for control or documentation purposes.

In this case, the breast milk is usually fed directly from the container to the premature or very premature infant in accordance with the present method. The container is thus made available for feeding the breast milk at the place of feeding. Thus, a fluidic connection is typically established between the container and the digestive tract of the premature or very premature infant when the breast milk is fed. Thus, the container is preferably fluidically connected to a feeding probe for feeding. This is usually located at the end of a tube provided between the feeding tube and the container and connected to the container. It is true that the organs of the very premature or premature infant, and in particular their digestive tract, are not yet mature and cannot absorb the entirety of their fluid and nutritional requirements by oral means from birth, so that an infusion is usually placed immediately after birth, through which parenteral nutrition can be carried out. This provides them with sugar, proteins, fats, minerals and vitamins. However, it should be fed breast milk in parallel and as soon as possible to stimulate the digestive tract. Breast milk has numerous properties that are beneficial for premature infants and is much better tolerated and digested by the immature intestine than artificial infant formula. This is where the invention particularly starts and aims to make the controlled feeding of expressed breast milk via a small feeding tube, which has been pushed through the nose into the stomach, safer and more reliable. The container identifier to the container and, if applicable, the storing of at least one specific property of the breast milk is preferably carried out in a form of a blockchain, which allows the documentation and tracking of the breast milk from pumping to feeding.

Breast milk used in a previously mentioned method may also be administered by breast-feeding mothers as a donation to an infant other than their own. The present invention aims to incentivize the donation activity of such donors as third parties and to facilitate the safe donation of breast milk. Thus, the present invention proposes an apparatus for handling breast milk which has an accommodation for the container containing the breast milk. The apparatus furthermore has a closing device for sealing the container. Sealing usually takes place in an automated manner. Particularly preferably, sealing takes place by sealing a flexible bag that forms the container. The apparatus also typically has a system for treating the breast milk contained in the container to improve the shelf life of the breast milk. Such treatment may be, for example, pasteurization by means of UV irradiation. The apparatus is further adapted to determine the amount of breast milk contained in the container. This determination can be carried out by detecting the volume contained in the container and/or by weighing. In this process, the tare weight of the container is usually communicated to the apparatus, for example by reading a container identifier provided on the container, which also contains information about the tare weight of the container. This allows the amount of breast milk contained in the container to be accurately determined. In addition to a reading device for reading out the container identifier provided on the container, the apparatus may also include a system for attaching a container identifier to the container. This system may save, as part of the container identifier, information relating to the properties of the breast milk which is determined in the apparatus. Additionally, the apparatus includes an interface for transmitting information to an online memory, wherein the information characterizes the container identifier or at least the determined amount of breast milk in the container. The apparatus may further have an apparatus identification provided with the container identifier at the interface for storage in the online memory. Such an apparatus identification allows a specific donation of breast milk to be associated with a specific apparatus and thus with a specific donor. Via this apparatus identification or identification of the donor, it is possible to transmit information about the property of the donated breast milk back to the donor and thus to incentivize further donations. In this context, the feedback is usually provided via a mobile device held by the donor, which is or will be connected to the online memory in terms of data, in order to transmit information about the property, for example the amount of donated milk, to the online memory. In this way, close-meshed and simple care of the donor is possible, as is monitoring of the quality of the donor's breast milk, if necessary. Thus, for example, results of a chemical analysis of the breast milk can be returned to the donor, which indicate a questionable quality of the breast milk and/or an illness in the donor, for example mastitis. If the donor is breast-feeding her own child, this type of feedback also allows qualitative monitoring of the breast milk given to her own infant.

The apparatus is usually also adapted to provide a time indication together with the container to the interface. This time indication allows an evaluation of the freshness of the milk. The time indication represents the day, if applicable, together with the time at which the breast milk was pumped and transferred to the container.

To feed the breast milk, the container is preferably subjected to an external pressure. In the case of a container with a rigid outer wall, the internal pressure is applied to the container so that the column of liquid provided in the container is expelled from the container. In the preferred configuration of the container in the form of a flexible bag, the container may be externally pressurized with hydrostatic pressure. Similarly, it is possible to apply pressure mechanically to the flexible container by means of a plunger or the like in order to discharge the breast milk contained in the bag. In this process, the external pressure is typically controlled so that the amount of breast milk discharged is set at the desired continuous or discontinuous volume or mass flow rate corresponding to the desired rate for feeding the premature or very premature infant. Alternatively, the breast milk can also be administered from the container in the manner of an infusion by gravity and under control of the volume flow as in the case of an infusion. As a rule, it is essential that the breast milk is administered from the container constantly and in a controlled manner, as required.

The previously discussed possibility of chemical analysis requires the drawing of a sample from the container. For this purpose, a flexible bag forming the container usually has a specially partitioned chamber for sampling and/or a resealable one-way valve closed under sterile conditions and adapted only for administering breast milk. Alternatively, the container may also have a separate cap for sampling. Such separate chambers or closures are usually formed on the flexible bag by sealing the flexible film material of the bag and/or sealing the closure, for example partitioning off a sample chamber by two spaced apart transverse sealing seams in the area of the grommet-shaped wall segments of the bag forming the entrance to the bag.

The method is preferably carried out as follows: A donor fluidically connects her breast pump to the container so that when the breast pump is operated, the container is filled with the breast milk to be donated.

The container is then inserted by the donor into the apparatus, which preferably carries out the further steps fully automatically, namely seals the container and, if necessary, divides off a sample chamber, treats the breast milk to improve its shelf life, determines the amount of breast milk contained in the container, and makes the corresponding donation contained in the container identifiable by means of the indication identifier and saves this identification in an online memory, if necessary together with further information on the properties of the breast milk. The apparatus may have a WLAN or LAN interface for this purpose. Storage is also usually fully automated after the apparatus has been set up.

The container is then delivered to a hospital, for example, and stored there. The information determined by the apparatus on the properties of the breast milk is sent in parallel to the donor's mobile device. At the hospital's incoming goods department, the container is usually scanned, i.e. the container identifier, which may be a bag identifier is read in or information on the properties of the breast milk inscribed in the container identifier, which may be the bag identifier, is read out. This information is saved in a central storage facility at the hospital. If necessary, a sample is taken from the container and chemically analyzed. The analysis result is also saved to the container identifier and thus to the container with the breast milk. In particular, if there are any abnormalities, the donor is informed again.

In the course of feeding, the breast milk in the container can not only be heated, but also enriched with nutrient concentrates, so-called fortifiers, or with medications. Feeding takes place directly from the container by applying pressure to the container.

Obviously, the method according to the invention allows for a hygienically perfect donation of breast milk, wherein the method supplies the breast milk in the best possible way for subsequent feeding, since only one single container is used for pumping and for feeding. The container identifier allows the container and the breast milk it contains to be identified uniquely, if necessary supplemented by information on properties such as quantity, age and/or chemical ingredients or modifications by fortifiers and/or medications. Since the corresponding information is saved as data on the container, this data can be read into a feeding plan at the time of feeding, documenting the nutrition of the premature or very premature infant.

The containers are usually supplied to the donors under germ-free or sterile conditions and are removed from sterile outer packaging under such conditions, so that germ-free or low-germ handling of the breast milk is possible at all times and continues until feeding.

1. A method for treating breast milk, in which
a breast pump is applied to the female breast;
the breast pump is operated to express breast milk and transfer it to a container;
the container is sealed,
a container identifier is generated that identifies the container and is or will be assigned to the container, and
the container is made available for feeding breast milk.

2. The method according to claim 1, wherein the container is or will be provided with a machine-readable container identifier identifying the container;
at least one property of the breast milk contained in the container is saved to the container identifier in a memory, and
the container identifier is read in or out at a transfer point where the breast milk contained in the container is at least one of being stored or consumed to output the at least one property of the breast milk contained in the container.

3. The method according to claim 1, wherein the breast milk is discharged directly from the container during feeding.

4. The method according to claim 1, wherein the container for feeding is connected to a tube comprising a feeding probe.

5. The method according to claim 1, wherein a flexible bag is used as container.

6. The method according to claim 2, wherein the container, after the breast milk has been expressed, is transferred to an apparatus by which the container is sealed, by which the breast milk is treated to improve the shelf life in the container and the amount of breast milk contained in the container is determined, by which a container identifier provided on the container is read out or the container is provided with a container identifier, and by which at least the determined amount is provided as a property together with the container identifier for saving in the memory.

7. The method according to claim 6, wherein at least one of a apparatus device identification or a time indication is provided by the apparatus together with the container identifier in the memory for saving in the memory.

8. The method according to claim 1, wherein the container is subjected to an external pressure during feeding for expelling the breast milk and that the external pressure is regulated for setting the expelling amount of breast milk.

9. The method according to claim 1, wherein at least one of nutrients or medications are added to the breast milk before feeding.

10. The method according to claim 1, wherein the breast milk is treated before feeding, in particular at least one of being filtered or concentrated.

11. The method according to claim 2, wherein a sample is drawn from the container and analyzed and that the result of the analysis is saved in the memory assigned to the container identifier.

12. The method according to claim 2, wherein at least one property saved to the breast milk in the memory is sent to a mobile device deposited in the memory as the mobile device of the donor of the breast milk.

13. An apparatus for treating breast milk comprising an accommodation for a container containing breast milk and a closing device for sealing the container, a system for treating the breast milk contained in the container to improve the shelf life of the breast milk, a device for determining the amount of breast milk contained in the container, a reading device for reading out a container identifier provided on the container or attaching device for attaching a container identifier to the container and an interface for transmitting information to a memory characterizing the container identifier and the determined amount of breast milk.

14. An apparatus according to claim 13, wherein the apparatus is adapted to provide at least one of an apparatus identification or a time indication together with the container identifier at the interface for saving in the memory.
